(19) United States
(12) Patent Application Publication
(10) Pub. No.: US 2009/0198135 A1
(43) Pub. Date: Aug. 6, 2009

(54) ULTRASONIC DIAGNOSTIC APPARATUS

(76) Inventors: Koji Yanagihara, Tokyo (JP); Shinichi Amemiya, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 12/363,223

(22) Filed: Jan. 30, 2009

(30) Foreign Application Priority Data
Jan. 31, 2008 (JP) 2008-020160

(51) Int. Cl. A61B 8/14; A61G 13/00

(52) U.S. Cl. 600/443; 5/601

(57) ABSTRACT

An ultrasonic diagnostic apparatus includes an operating device for an operator to input an instruction, an image display device for displaying an ultrasonic image, a processing device which controls producing the ultrasonic image based on an echo signal obtained by sending and receiving a ultrasonic wave and displaying it, and a table on which a patient lies. Said operating device is provided separately from said image display device, said processing device, and said table. Said table, said image display device, and said processing device are integrally provided on the side where said operating device is not placed with respect to the table.

### CROSS REFERENCE TO RELATED APPLICATIONS

This application claims the benefit of Japanese Patent Application No. 2008-020160 filed Jan. 31, 2008, which is hereby incorporated by reference in its entirety.

### BACKGROUND OF THE INVENTION

The embodiments described herein relate to an ultrasonic diagnostic apparatus that produces an ultrasonic image based on an echo signal obtained by sending an ultrasonic wave.

### BACKGROUND ART

A conventional ultrasonic diagnostic apparatus includes: an operating device for an operator to input an instruction; an image display device for displaying an ultrasonic image; and a processing device which controls producing ultrasonic image data based on an echo signal obtained by sending an ultrasonic wave and displaying it. The operating device, the image display device, and the processing device can move as one unit (see, for example, Japanese Unexamined Patent Publication No. 2003-339708)

### TECHNICAL PROBLEM

In order to position the operating device within the reach of the operator, the conventional ultrasonic diagnostic apparatus is placed on the side of the operator with respect to a bed on which a patient receiving an ultrasonic examination lies. However, since the operating device, the image display device, and the processing device are integrally provided, when the large conventional ultrasonic diagnostic apparatus is placed on the side of the operator with respect to the bed, a considerable part of a space where the operator works is occupied.

Further, depending on a portion to be examined by ultrasonic waves, it may be difficult for an examination technician to examine the patient who is lying on the bed. In such a case, the patient sometimes changes his or her posture to be examined easily, which may give a burden to the patient. Further, among the patients, there are some people who are old or injured and find it difficult to get on or off the bed by themselves.

### BRIEF DESCRIPTION OF THE INVENTION

In view of the above, embodiments of the invention provide an ultrasonic diagnostic apparatus which makes it possible to effectively use a space in a room in which the ultrasonic diagnostic apparatus is installed.

Further, embodiments of the invention provide an ultrasonic diagnostic apparatus which makes it possible to effectively use a space in a room in which the ultrasonic diagnostic apparatus is installed, to allow a patient to be at a position, without being given a burden, where an examination technician can easily examine the patient according to a portion to be examined by ultrasonic waves, and to allow the patient to easily get on and off a table which the patient lies on.

In a first aspect of the invention, there is provided an ultrasonic diagnostic apparatus including: an operating device for an operator to input an instruction; an image display device for displaying an ultrasonic image; a processing device which controls producing the ultrasonic image based on an echo signal obtained by sending an ultrasonic wave and displaying it; and a table on which a patient lies. The operating device is formed separately from the image display device, the processing device, and the table. Further, on the side where the operating device is not placed with respect to the table, there are integrally provided the table, the image display device, and the processing device.

In a second aspect of the invention, there is provided an ultrasonic diagnostic apparatus according to the first aspect of the invention, wherein there are provided, on the table, the processing device on the side where the operating device in not provided, and the image display device at the processing device.

In a third aspect of the invention, there is provided an ultrasonic diagnostic apparatus according to the first aspect, wherein, on the table, the image display device and the processing device are provided on the side where the operating device is not placed.

In a fourth aspect of the invention, there is provided an ultrasonic diagnostic apparatus according to any one of the first to third aspects, wherein the processing device has a portion whose height from a floor is lower than the table and a thickness of the portion is 15 cm or less.

In a fifth aspect of the invention, there is provided an ultrasonic diagnostic apparatus according to the third aspect, wherein said image display device is provided at the table via a mounting part; wherein each of the mounting part and the processing device has a portion whose height from the floor is lower than the table; and wherein a thickness of the portion is 15 cm or less.

In a six aspect of the invention, there is provided an ultrasonic diagnostic apparatus according to any one of the first to fifth aspects, wherein a probe for sending and receiving ultrasonic waves and the processing device have radio communication units, respectively, for mutual radio communication.

In a seventh aspect of the invention, there is provided an ultrasonic diagnostic apparatus according to any one of the first to fifth aspects, wherein a probe body of the probe for sending and receiving ultrasonic waves and the processing device are connected through a cable; and wherein there is provided a cable receiving section for receiving the cable on the side where said operating device is not placed with respect to the table.

In an eighth aspect of the invention, there is provided an ultrasonic diagnostic apparatus according to the seventh aspect, wherein said cable receiving section has a cable receiving case having a cable receiving space therein and being integrally formed with the table; wherein the cable receiving case has a portion whose height from the floor is lower than the table; and wherein a thickness of the portion is 15 cm or less.

In a ninth aspect of the invention, there is provided an ultrasonic diagnostic apparatus according to the eighth aspect, wherein the processing device is integrally provided in an upper portion of the cable receiving case.

In a tenth aspect of the invention, there is provided an ultrasonic diagnostic apparatus according to any one of the first to ninth aspects, wherein the shape of the table is changeable.

In an eleventh aspect of the invention, there is provided an ultrasonic diagnostic apparatus according to the tenth aspect, wherein the shape of the table can be changed into the shape of a bed and the shape of a chair.

In a twelfth aspect of the invention, there is provided an ultrasonic diagnostic apparatus according to any one of the first to eleventh aspects, wherein the table has a height-adjusting part for adjusting a height of a mounting platform, from the floor, of the table on which a patient lies.

In a thirteenth aspect of the invention, there is provided an ultrasonic diagnostic apparatus according to any one of the first to twelfth aspects, wherein the table has a grip part that the patient holds onto.

According to the ultrasonic diagnostic apparatus of the first aspect of the invention, the operating device to be placed on the side of the operator with respect to the table is provided separately from the image display device, the processing device, and the table and, on the side where the operating device is not placed with respect to the table, there are integrally provided the table, the image display device, and the processing device. As a result of this, the image display device and the processing device are eliminated from the space where the operator works, making the space larger than before. Thus, the space in the room in which the ultrasonic diagnostic apparatus is installed can be used effectively.

According to the ultrasonic diagnostic apparatus of the second aspect of the invention, there are provided, on the table, the processing device on the side where the operating device is not provided and the image display device at the processing device, and the table, the processing device, and the image display device are provided integrally. As a result of this, the image display device and the processing device are eliminated from the space where the operator works, making the space larger than before. Thus, the space in the room in which the ultrasonic diagnostic apparatus is installed can be used effectively.

According to the ultrasonic diagnostic apparatus of the third aspect of the invention, there are provided, on the table, the processing device and the image display device on the side where the operating device is not provided, and the table, the image display device, and the processing device are provided integrally. As a result of this, the image display device and the processing device are eliminated from the space where the operator works, making the space larger than before. Thus, the space in the room in which the ultrasonic diagnostic apparatus is installed can be used effectively.

According to the ultrasonic diagnostic apparatus of the fourth aspect of the invention, a portion of the processing device whose height from the floor is lower than the table has a thickness of 15 cm or less. Therefore, it is possible to set a spacing between the table and a wall to 16 cm, for example, and to place the table as close as possible to the wall. As a result of this, the space where the operator works becomes larger than before, making it possible to effectively use the space in the room in which the ultrasonic diagnostic apparatus is installed.

According to the ultrasonic diagnostic apparatus of the fifth aspect of the invention, each of portions of the mounting part and the processing device lower than the table has a thickness of 15 cm or less. Therefore, it is possible to set a spacing between the table and the wall to 16 cm, for example, and to place the table as close as possible to the wall. As a result of this, the space where the operator works becomes larger than before, making it possible to effectively use the space in the room in which the ultrasonic diagnostic apparatus is installed.

According to the ultrasonic diagnostic apparatus of the sixth aspect of the invention, the cable for connecting the probe with the processing device can be eliminated, preventing the cable from being in the way.

According to the ultrasonic diagnostic apparatus of the seventh aspect of the invention, the cable can be received in the cable receiving section, preventing the cable from being in the way.

According to the ultrasonic diagnostic apparatus of the eighth aspect of the invention, the thickness of the portion of the cable receiving case lower than the table is 15 cm or less. Therefore, it is possible to set the spacing between the table and the wall to 16 cm, for example, and to place the table as close as possible to the wall. As a result of this, the space where the operator works becomes larger than before, making it possible to effectively use the space in the room in which the ultrasonic diagnostic apparatus is installed.

According to the ultrasonic diagnostic apparatus of the ninth aspect of the invention, the processing device is provided above the cable receiving case whose portion lower than the table is 15 cm thick or less. Therefore, if the spacing between the table and the wall is set to 16 cm, for example, the table can be placed as close as possible to the table. As a result of this, the space where the operator works becomes larger than before, making it possible to effectively use the space in the room in which the ultrasonic diagnostic apparatus is installed.

According to the ultrasonic diagnostic apparatus of the tenth aspect of the invention, the same advantages as those of the first to ninth aspects of the invention can be obtained. Also, the shape of the table on which the patient lies is changeable. Therefore, by adjusting the shape of the table according to a portion to be given an ultrasonic examination, the patient can be moved to a height on a position where the examination technician can carry out an examination easily without giving a burden to the patient. Further, change in the shape of the table allows the patient to get on and off the table easily.

According to the ultrasonic diagnostic apparatus of the eleventh aspect of the invention, the shape of the table can be changed into the shape of the bed and the shape of the chair, making it possible to move the patient to a height or a position where the examination technician can carry out the examination easily without giving a burden to the patient. Further, it becomes possible for the patient to get on and off the table easily.

According to the ultrasonic diagnostic apparatus of the twelfth aspect of the invention, the same advantages as those of the first to eleventh aspects can be obtained. Furthermore, by adjusting the height of the mounting plane of the table from the floor by use of the height-adjusting part, the patient can be positioned at the height where the examination technician can easily carry out the examination according to the portion to be examined by ultrasonic waves. Also, the patient can easily get on and off the table.

According to the ultrasonic diagnostic apparatus of the thirteenth aspect of the invention, the table has the grip part. Therefore, for example, when the patient gets on or off the table, or gets up from a lying position on the table, the patient can easily do so by holding onto the grip part.

Further objects and advantages of the present invention will be apparent from the following description of the preferred embodiments of the invention as illustrated in the accompanying drawings.

### BRIEF DESCRIPTION OF THE DRAWINGS

FIG. 1 is a partially broken side view showing an exemplary ultrasonic diagnostic apparatus according to a first embodiment.

FIG. 2 is a front view of the ultrasonic diagnostic apparatus shown in FIG. 1.

FIG. 3 is a plan view of the ultrasonic diagnostic apparatus shown in FIG. 1.

FIG. 4 is a front view of the ultrasonic diagnostic apparatus whose table is in the shape of a chair.

FIG. 5 is a side view showing an exemplary ultrasonic diagnostic apparatus according to a second embodiment.

FIG. 6 is a front view of the ultrasonic diagnostic apparatus shown in FIG. 5.

FIG. 7 is a plan view of the ultrasonic diagnostic apparatus shown in FIG. 5.

FIG. 8 is a partially broken side view showing an exemplary ultrasonic diagnostic apparatus according to a third embodiment.

FIG. 9 is a front view of the ultrasonic diagnostic apparatus shown in FIG. 8.

FIG. 10 is a plan view of the ultrasonic diagnostic apparatus shown in FIG. 8.

FIG. 11 is a partially broken enlarged side view of the vicinity of the opening of the ultrasonic diagnostic apparatus shown in FIG. 8.

FIG. 12 is an enlarged front view of the vicinity of the opening of the ultrasonic diagnostic apparatus shown in FIG. 8.

FIG. 13 is an enlarged side view of the vicinity of the movable pulley, wherein the cover attached to the cable receiving case of the ultrasonic diagnostic apparatus of FIG. 8 is slid downwards to expose the inside.

FIG. 14 is an enlarged side view of the vicinity of the movable-pulley lock, wherein the movable-pulley lock is released.

FIG. 15 is an enlarged side view of the vicinity of the movable-pulley lock, wherein the movable-pulley lock is working.

FIG. 16 is an enlarged side view of the vicinity of the movable pulley, wherein the cable lock is released.

### DETAILED DESCRIPTION OF THE INVENTION

Hereinbelow, embodiments of the invention will be described in detail with reference to drawings.

### Embodiment 1

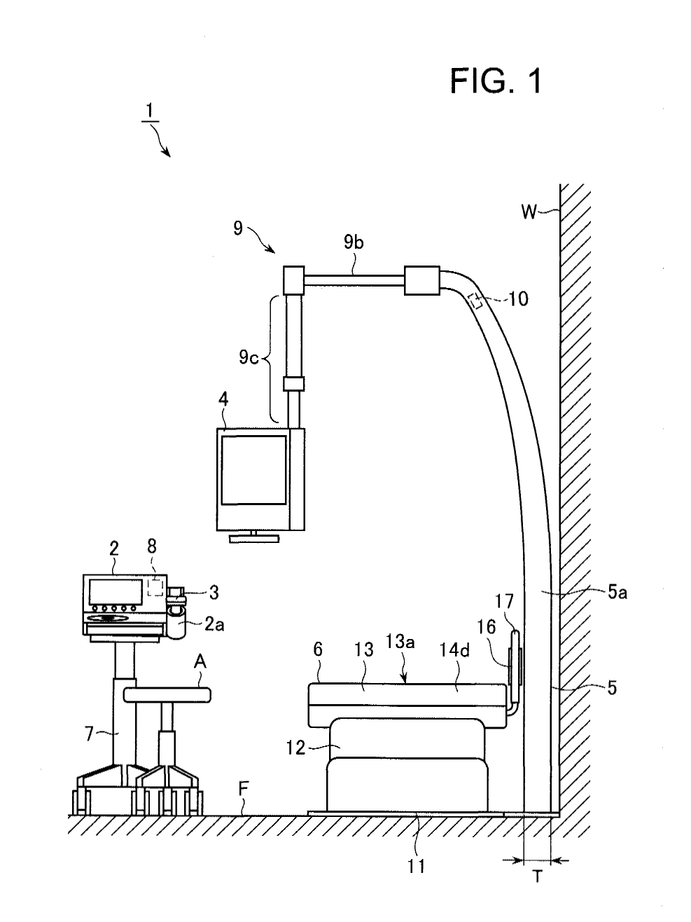

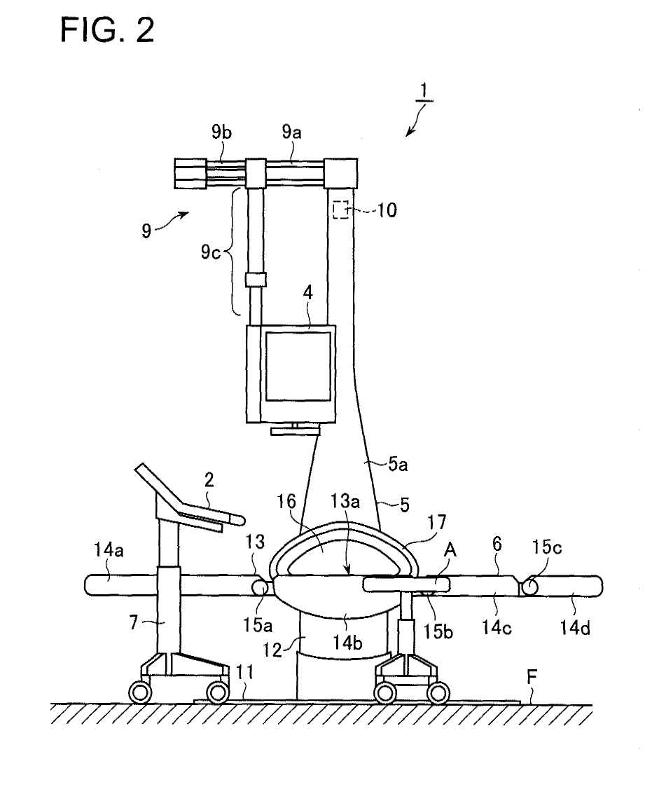

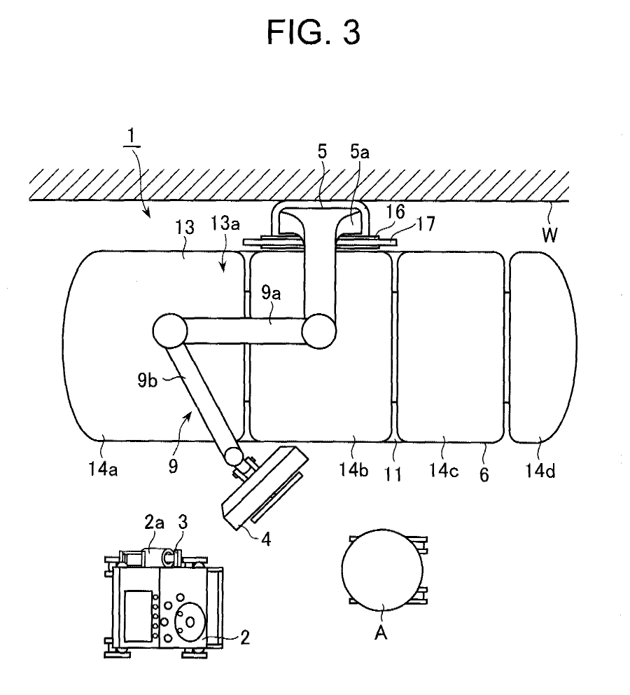

First, Embodiment 1 of the invention will be described. FIG. 1 is a partially broken side view showing an ultrasonic diagnostic apparatus according to Embodiment 1 of the invention, FIG. 2 is a front view of the ultrasonic diagnostic apparatus shown in FIG. 1, and FIG. 3 is a plan view of the ultrasonic diagnostic apparatus shown in FIG. 1.

The ultrasonic diagnostic apparatus 1 includes: an operating device 2 for an operator to input an instruction; a probe 3 for sending and receiving ultrasonic waves; an image display device 4 for displaying an ultrasonic image; a processing device 5 which controls driving the probe 3 based on the instruction by the operator, producing the ultrasonic image based on a received echo signal, and displaying it on the image display device 4; and a table 6 on which a patient lies.

The operating device 2 is provided separately from the image display device 4 and the processing device 5. Further, the operating device 2 is provided on a movable part 7 with a caster, and is placed on the side where a chair A on which the operator sits is placed with respect to the table 6. On a side portion of the operating device 2, there is provided a probe holder 2a in a cylindrical shape with a bottom that accommodates the probe 3 (not shown in FIG. 2). Incidentally, in the present embodiment, as described later, the probe 3 communicates with the processing device 5 by radio.

Further, the operating device 2 has a radio communication unit 8 on the side of the operating device for radio communication with the processing device 5. When the operator inputs an instruction through the operating device 2, the instruction signal is sent to the processing device 5 through the radio communication unit 8 on the operating device side. Thus, by allowing the operating device 2 and the processing device 5 to communicate with each other by radio, a cable for connecting these devices can be eliminated, the operating device 2 can freely be moved by the movable part 7, and the connection cable is prevented from being in the way.

The image display device 4, the processing device 5, and the table 6 are provided integrally on the side (the side of the wall W in the present embodiment) where the operating device 2 is not placed with respect to the table 6. In the present embodiment, the processing device 5 is provided on a base plate 11 (to be described later) of the table 6, and the image display device 4 is provided at the processing device 5.

The image display device 4 is provided at the processing device 5 via an arm 9.

The arm 9 includes: a first horizontal arm 9a extending horizontally from the processing device 5; a second horizontal arm 9b extending horizontally from the first horizontal arm 9a; and an up-and-down arm 9c extending vertically from the second horizontal arm 9b. The image display device 4 is attached to a lower end of the up-and-down arm 9c.

The first horizontal arm 9a rotates horizontally at a joint with the processing device 5. Further, the second horizontal arm 9b rotates horizontally at a joint with the first horizontal arm 9a. The length of the up-and-down arm 9c is variable. Also, the up-and-down arm 9c rotates horizontally at a joint with the second horizontal arm 9b. As a result of this, the image display device 4 can be moved to an appropriate position for viewing according to a posture of an examination technician, and the examination technician can carry out an examination in a natural posture.

The image display device 4 rotates vertically at a joint with the up-and-down arm 9c so that the angle of its screen can be adjusted.

The processing device 5 has a processing-device case 5a in a columnar shape whose width in a front view increases from its top towards bottom. In a lower portion inside the processing-device case 5a, there is accommodated a processing part which controls producing an ultrasonic image and displaying it. Data of the ultrasonic image produced by the processing part is sent to the image display device 4 through a cable (not shown) arranged inside the arm 9.

As seen from the side, an upper portion of the processing-device case 5a is curved towards the table 6. Further, a thickness T of the processing-device case 5a is approximately 10.0 centimeters (cm) at the maximum. As described above, the thickness T of the processing-device case 5a is at most approximately 10.0 cm, and the curving of the processing-device case 5a starts from a portion above a safety handrail 17 (to be described) of the table 6. Therefore, a spacing between the table 6 and the wall W can be set to approximately 11.0 cm, for example, so that the table 6 can be placed as close as possible to the wall. Further, the curving of the processing-device case 5a starts from a position where the safety handrail 17 (to be described) does not contact the processing-device case 5a even when a mounting platform 13 (to be described) of the table 6 whose height from the floor F can be adjusted is at the uppermost position.

In this regard, according to the invention, the thickness T is not limited to approximately 10.0 cm. However, in terms of minimizing the spacing between the table 6 and the wall W, the thickness T is preferably approximately 15.0 cm or less.

Inside the processing-device case 5a, there is provided a processing-device side communication unit 10. The processing-device side communication unit 10 and the probe 3 communicate with each other by radio waves. By the processing-device side radio communication unit 10, a control signal for allowing an ultrasonic wave to be sent from the side of the processing device 5 to the probe 3 is sent by radio. At the same time, when an echo signal obtained by the probe 3 is sent to the side of the processing device 5 by radio, the echo signal is received by the processing-device side radio communication unit 10. Further, the processing-device side radio communication unit 10 also communicates with the operating-device side radio communication unit 8 by radio.

The table 6 includes: a rectangular base plate 11 placed on the floor F; a lift 12 provided on the base plate 11; and the mounting platform 13 provided on the lift 12. The patient is mounted on a mounting plane 13a of the mounting platform 13 to be given an ultrasonic examination.

On the base plate 11, a rail (not shown) is provided in a longitudinal direction (right-and-left direction as seen from the front) and, on the rail, the lift 12 is provided in a freely movable manner. The lift 12 is moved along the rail by a motor.

The lift 12 can be extended and contracted vertically. The extension and contraction of the lift 12 is also driven by the motor. By use of the lift 12, the height of the mounting plane 13a from the floor F can be adjusted. The lift 12 is an example of the embodiment of the height-adjusting part of the invention.

The mounting platform 13 includes a first plate-like member 14a, a second plate-like member 14b, a third plate-like member 14c, and a fourth plate-like member 14d. A joint between the first plate-like member 14a and the second plate-like member 14b has a first rotary shaft 15a. About the first rotary shaft 15a, the first plate-like member 14a can be rotated upwards with respect to the second plate-like member 14b. Further, a joint between the second plate-like member 14b and the third plate-like member 14c has a second rotary shaft 15b. About the second rotary shaft 15b, the third plate-like member 14c can be rotated downward(s) with respect to the second plate-like member 14b. Still further, a joint between the third plate-like member 14c and the fourth plate-like member 14d has a third rotary shaft 15c. About the third rotary shaft 15c, the fourth plate-like member 14d is rotated upwards. Also, each of the plate-like members 14a to 14d can be fixed at a position where it is rotated. By the rotation of the first plate-like member 14a, the third plate-like member 14c, and the fourth plate-like member 14d, the shape of the table 6 can be changed into the shape of a bed and the shape of a chair.

On a side portion of the second plate-like member 15b, there are provided a side plate 16 and a safety handrail 17. The safety handrail 17 is an example of the embodiment of the grip part of the invention. With the safety handrail 17 mounted, for example, when the patient gets on and off the table 6 or gets up from a lying position on the table 6, by holding onto the safety handrail 17, it becomes possible for the patient to get on and off the table 6 or get up easily.

According to the ultrasonic diagnostic apparatus 1 of the present embodiment, the operating device 2 which has to be placed on the side of the operator with respect to the table 6 is provided separately from the image display device 4, the processing device 5, and the table 6 and, on the side where the operating device 2 is not placed with respect to the table 6, there are integrally provided the table 6, the image display device 4, and the processing device 5. As a result of this, the image display device 4 and the processing device 5 are eliminated from the space where the operator works, making the space larger than before. Thus, the space in the room in which the ultrasonic diagnostic apparatus 1 is installed can be used effectively.

Further, according to the present embodiment, the thickness T of the processing-device case 5a is approximately 10.0 cm at the maximum. Therefore, the spacing between the table 6 and the wall W can be set to approximately 11.0 cm, for example, so that the table 6 can be placed as close as possible to the wall W. As a result, the space where the operator works becomes larger than before, and the space in the room in which the ultrasonic diagnostic apparatus 1 is installed can be used effectively.

Further, the table 6, the image display device 4, and the processing device 5 are integrally provided, so that the table 6 is heavier than the image display device 4. Therefore, the table 6, the image display device 4, and the processing device 5 can be installed in a stable state. Furthermore, since those devices can be installed stably, the installation of the ultrasonic diagnostic apparatus 1 can be completed simply by placing the table 6, the image display device 4, and the processing device 5 at installation positions, which makes the installation easy. Further, when more stable installation is required, the base plate 11 of the table 6 may be fastened to the floor F with anchor bolts (not shown).

Further, the probe 3 and the processing device 5 communicate with each other by radio waves. Therefore, the cable for connecting the probe 3 with the processing device 5 can be eliminated, preventing the cable from being in the way.

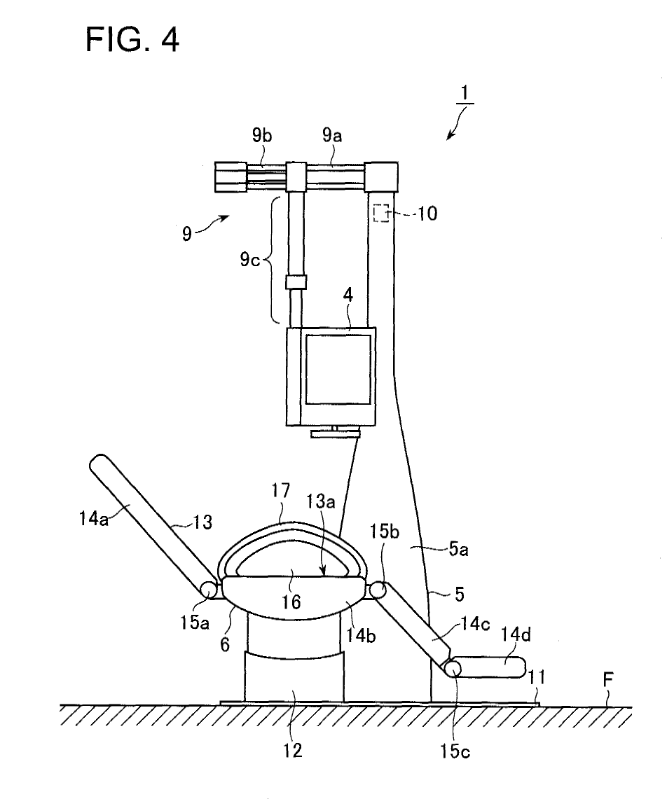

Furthermore, the table 6 in the shape of the bed where the mounting plane 13a of the mounting platform 13 is horizontal can be changed as shown in FIG. 4, for example, by rotating the first plate-like member 14a, the third plate-like member 14c, and the fourth plate-like member 14d into the shape of the chair. Also, in accordance with the posture of the patient during the examination, besides the shape of the chair, the shape of the table 6 can be changed differently by rotating the first plate-like member 14a, the third plate-like member 14c, and the fourth plate-like member 14d. As a result of this, without giving the patient a burden, the patient can be placed at a height or a position where the examination technician can carry out the examination easily. At the same time, the patient can get on and off the table 6 easily.

When the shape of the table 6 is changed, if the image display device 4 is behind the patient and the examination technician cannot see the image display device 4 well, the lift 12 which is movable on the base plate 11 horizontally may be moved so that the image display device 4 can be seen better. Incidentally, in FIG. 4, the lift 12 is positioned on the left with respect to a central portion of the base plate 11.

The height of the mounting plane 13a of the table 6 from the floor F can be adjusted by the lift 12. Therefore, the patient can be positioned at a height where the examination technician can carry out the examination easily according to a portion to be examined by ultrasonic waves. Further, the patient can get on and off the table 6 easily.

### Embodiment 2

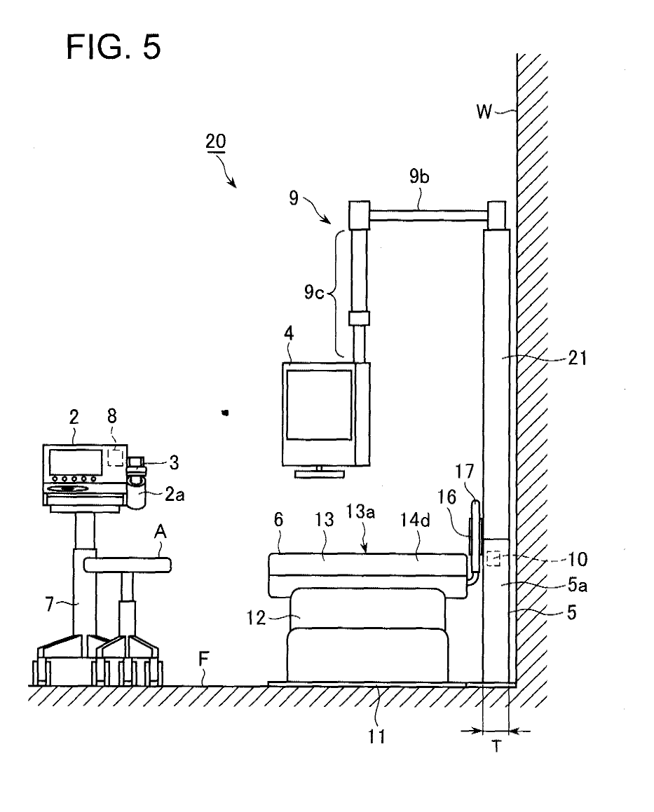

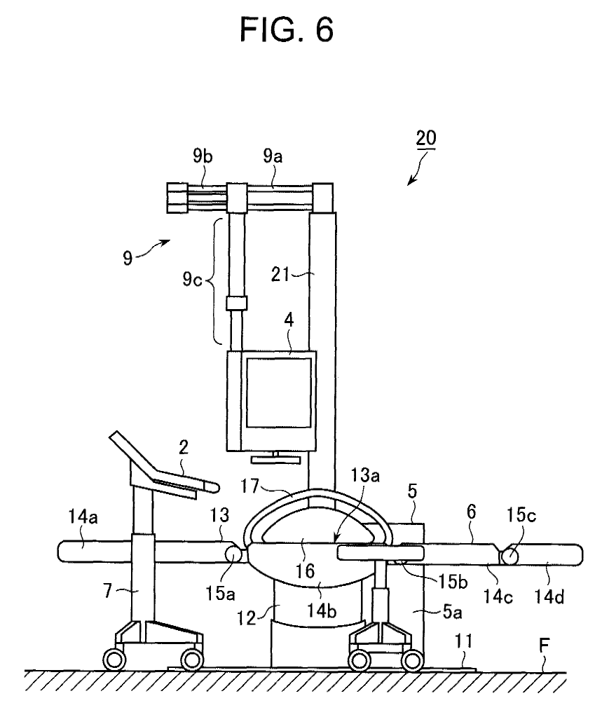

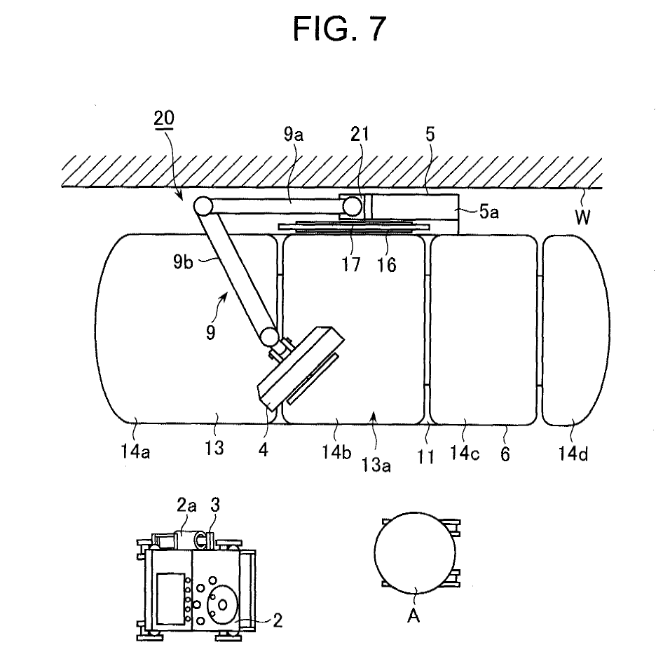

Next, with reference to FIGS. 5 to 7, Embodiment 2 of the invention will be described. FIG. 5 is a side view showing an ultrasonic diagnostic apparatus according to Embodiment 2 of the invention, FIG. 6 is a front view of the ultrasonic diagnostic apparatus shown in FIG. 5, and FIG. 7 is a plan view of the ultrasonic diagnostic apparatus shown in FIG. 5. In the drawings, like reference characters refer to like parts in Embodiment 1 and descriptions thereof will be omitted.

In the ultrasonic diagnostic apparatus 20 according to the present embodiment, there are provided a column support 21 and a processing device 5 in a row on a base plate 11 of the table 6. The column support 21 and the processing device 5 are provided on the side where the operating device 2 is not placed with respect to the table. On the column support 21, the image display device 4 is provided via the arm 9. Therefore, the image display device 4 is provided on the base plate 11 via the column support 21 and is, together with the processing device 5, provided integrally with the table 6. The column support 21 is an example of the embodiment of a mounting part of the invention.

According to the present embodiment, the processing-device case 5a of the processing device 5 is in the shape of a rectangular parallelpiped. The thickness T of the processing-device case 5a and the column support 21 is approximately 10.0 cm. As in Embodiment 1, according to the invention, the thickness T is not limited to 10.0 cm. However, in terms of minimizing the spacing between the table 6 and the wall W, it is desirable that T is approximately 15.0 cm or less. Inside the processing-device case 5a, there is provided a processing-device side radio communication unit 10. Further, from the processing-device case 5a, a cable (not shown) is pulled out, and the cable is connected with the image display device 4 through the column support 21 and the arm 9. By use of the cable, data of the ultrasonic image produced in the processing device 5 is sent to the image display device 4.

In the above ultrasonic diagnostic apparatus 20 of the present embodiment also, the table 6, the image display device 4, and the processing device 5 are provided separately from the processing device 2. Further, the table 6, the image display device 4, and the processing device 5 are provided integrally on the side where the operating device 2 is not placed with respect to the table 6. Therefore, as in Embodiment 1, the space in the room in which the ultrasonic diagnostic apparatus 20 is installed can be used effectively.

According to the present embodiment, the thickness T of the processing-device case 5a and the column support 21 is approximately 10.0 cm. Therefore, the spacing between the table 6 and the wall W can be set to approximately 11.0 cm, for example. Thus, the table 6 can be placed as close as possible to the wall W.

Also, according to the ultrasonic diagnostic apparatus 20 of the present embodiment, the same advantages as other advantages of Embodiment 1 can be obtained.

### Embodiment 3

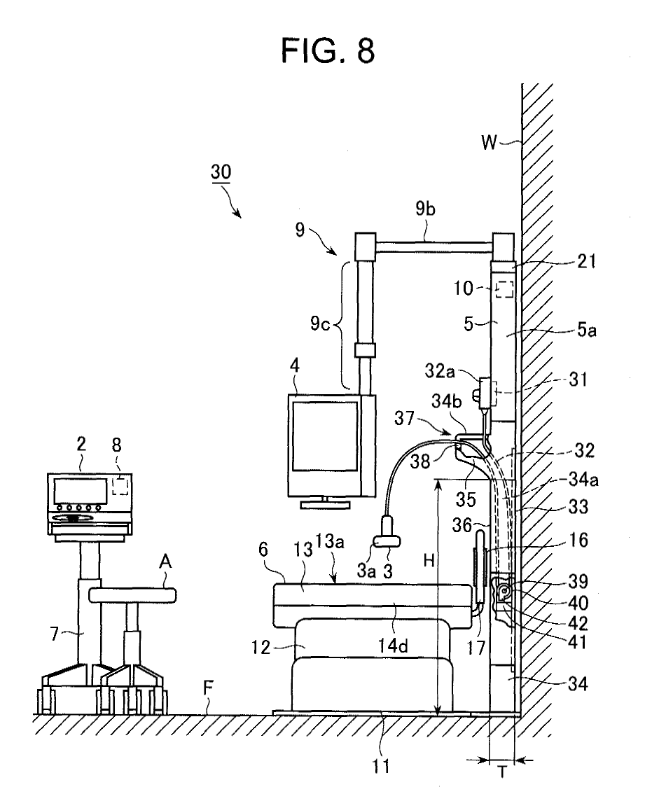

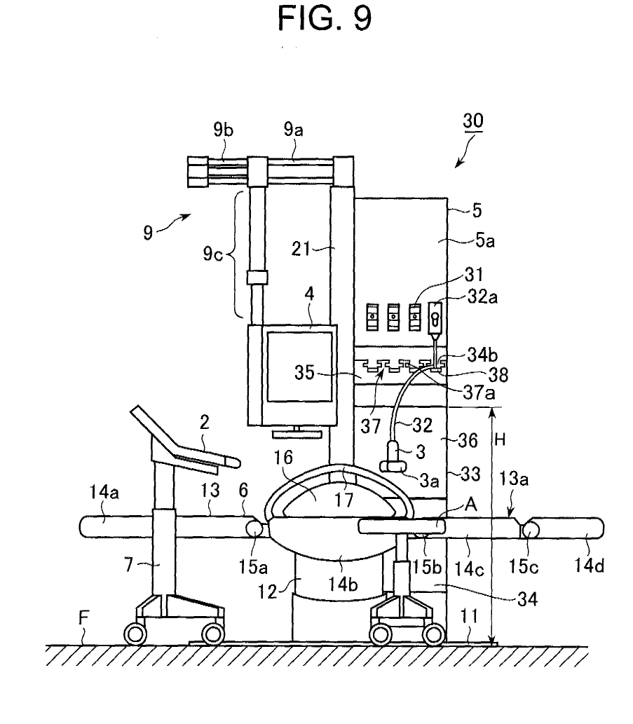

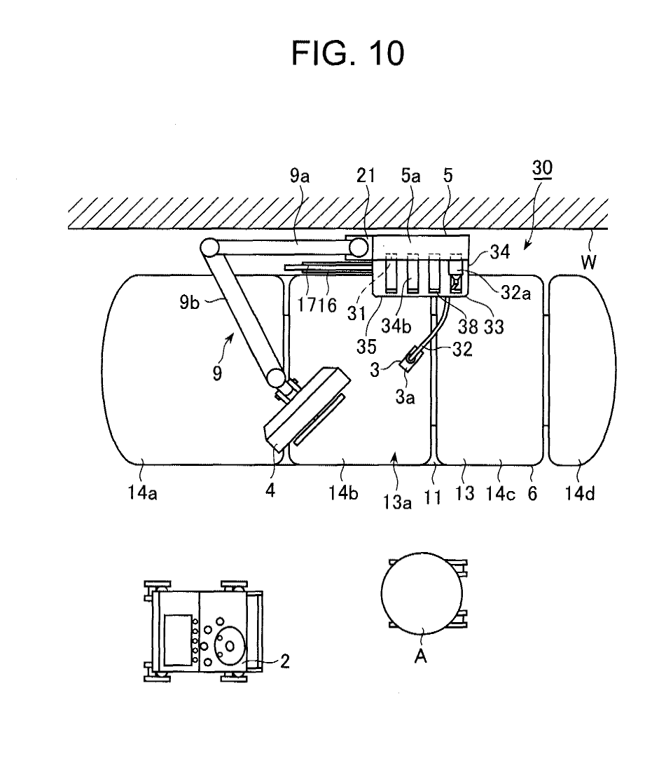

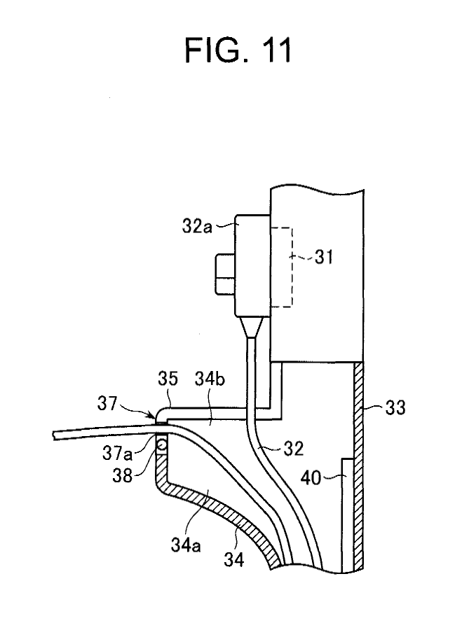

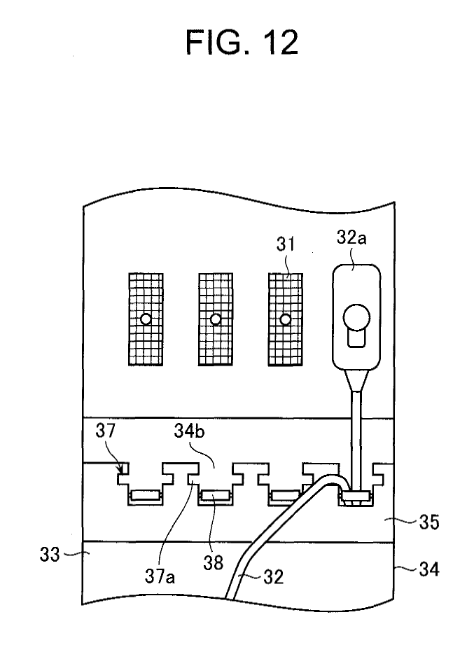

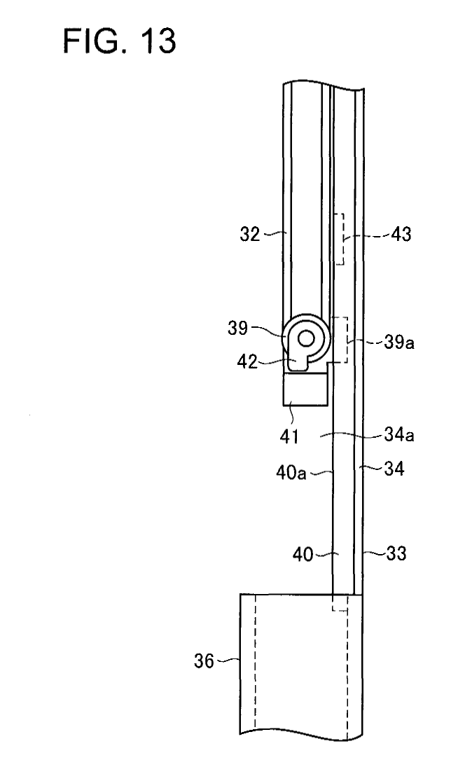

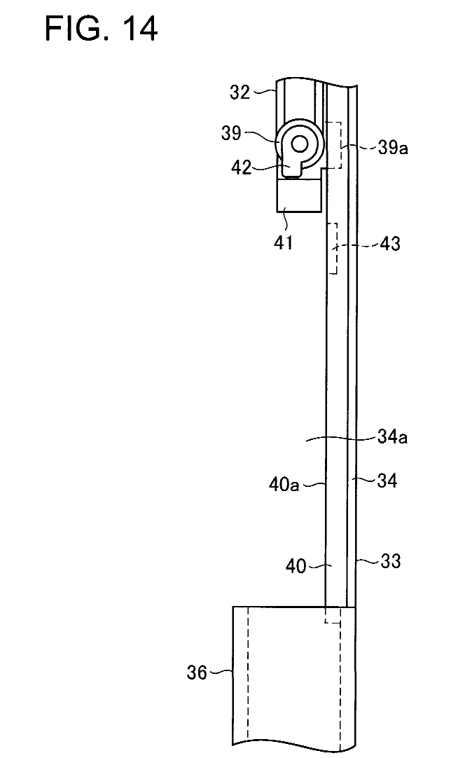

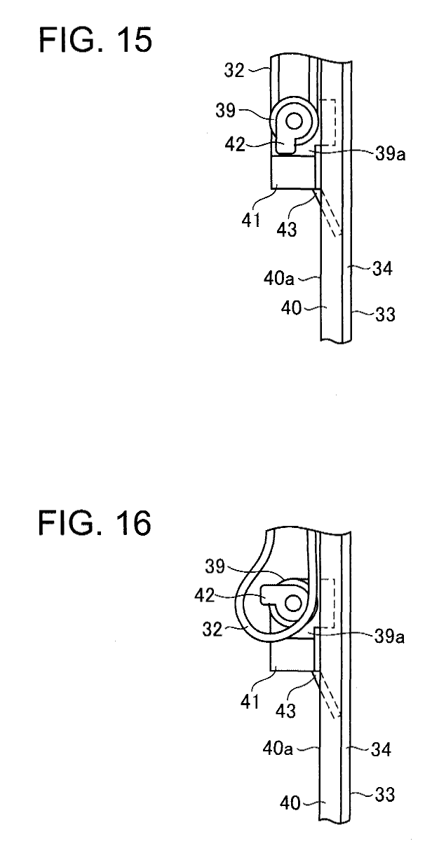

Next, with reference to FIGS. 8 to 16, Embodiment 3 of the invention will be described. FIG. 8 is a partially broken side view of the ultrasonic diagnostic apparatus according to Embodiment 3 of the invention, FIG. 9 is a front view of the ultrasonic diagnostic apparatus shown in FIG. 8, FIG. 10 is a plan view of the ultrasonic diagnostic apparatus shown in FIG. 8, FIG. 11 is a partially broken enlarged side view of the vicinity of the opening of the ultrasonic diagnostic apparatus shown in FIG. 8, and FIG. 12 is an enlarged front view of the vicinity of the opening of the ultrasonic diagnostic apparatus shown in FIG. 8. Further, FIG. 13 is an enlarged side view of the vicinity of a movable pulley in which a cover attached to the cable receiving case of the ultrasonic diagnostic apparatus shown in FIG. 8 is slid downwards to expose the inside, FIG. 14 is an enlarged side view of the vicinity of a movable-pulley lock (a state where the movable-pulley lock is released), FIG. 15 is an enlarged side view of the vicinity of the movable-pulley lock (a state where the movable-pulley lock is working), and FIG. 16 is an enlarged side view of the vicinity of the movable pulley (a state where the cable lock is released). In the drawings, like reference characters refer to like parts in Embodiments 1 and 2 and descriptions thereof will be omitted.

According to an ultrasonic diagnostic apparatus 30 of the present embodiment, as in Embodiment 2, the image display device 4 is provided on the base plate 11 of the table 6 via the column support 21. Further, the processing device 5 is integrally provided in an upper portion of a cable receiving unit 33 (to be described). In the present embodiment also, the processing-device case 5a of the processing device 5 is in the shape of a rectangular parallelpiped. In the processing-device case 5a, there is provided a probe connector 31 for the connection of a connector 32a of a cable 32 to be described for connecting a probe body 3a with the processing device 5.

In the present embodiment, the probe body 3a and the processing device 5 are connected through the cable 32. Further, there is provided the cable receiving unit 33 for receiving the cable 32 on the side where the operating device 2 is not placed with respect to the table 6. The cable receiving unit 33 is provided on the base plate 11 of the table 6. Therefore, the processing device 5 provided in the upper portion of the cable receiving unit 33 is integral with the table 6 via the cable receiving unit 33.

The cable receiving unit 33 is an example of the embodiment of the cable receiving section of the invention. The cable receiving unit 33 has a cable receiving case 34 where there is formed a cable receiving space 34a for receiving the cable 32 for connecting the probe body 3a with the processing device 5. The processing device 5 is provided integrally with the cable receiving case 34. The height of the cable receiving case 34 is approximately 150.0 cm. The thickness T of a portion of the cable receiving case 34 up to a height H of approximately 100.0 cm that is higher than that of the table 6 is approximately 10.0 cm. The thickness T is not limited to 10.0 cm in the invention. However, in terms of minimizing the spacing between the table 6 and the wall W, the thickness T is preferably approximately 15.0 cm or less. Further, a portion of the cable receiving case 34 between approximately 100.0 cm and approximately 130.0 cm in height is a projection 35, whose thickness is approximately 20.0 cm.

Further, the range where the mounting platform 13 goes up and down is set such that the safety handrail 17 does not contact the projection 35 even when the mounting platform 13 of the table 6 reaches the uppermost position.

Mounted on the cable receiving case 34 is a cover 36 that is vertically slidable. When the cover 36 is slid downwards, the inner portion of the cable receiving case 34 between approximately 65.0 cm and approximately 100.0 cm in height is exposed, allowing the operator to access the cable receiving space 34a. As a result, it becomes easier to do the maintenance work and, further, possible to remove the cable 32 of the probe 3 from the movable pulley 39 (to be described) and to mount the cable 32 on the movable pulley 39.

A portion of the projection 35 of the cable receiving case 34 between approximately 120.0 cm and approximately 130.0 cm in height is an opening 34b. The probe connector 31 is provided above the opening 34b, and the cable 32 of the probe 3 is received in the cable receiving space 34a through the opening 34b.

The cable 32 is bent in the shape of a U and is received in the cable receiving space 34a. With the cable 32 being received in this state, only one portion at the lower end (where the movable pulley 39 is hung) of the U-shaped cable 32 is bent, preventing the cable 32 from being damaged.

A cable stopper 37 is provided in the opening 34b. The cable stopper 37 is a slit 37a that is narrower than an outside diameter of the cable 32. By elastically deforming and inserting the cable 32 in the slit 37a, the cable 32 can be stopped in an engaged state. As a result of this, the cable 32 is prevented from going out of the opening 34b or being pulled into the cable receiving space 34b against the operator's will.

Moreover, in the opening 34b, there is provided a roller 38 which may contact an undersurface of the cable 32. Because of the roller 38, the cable 32 can smoothly comes in and goes out of the opening 34b.

Now, with reference to FIG. 13, an inner construction of the cable receiving case 34 will be described. FIG. 13 is the enlarged side view of the vicinity of the movable pulley 39 in which the cover 36 attached to the cable receiving case 34 is slid downwards and the inner portion of the cable receiving case 34 between approximately 65.0 cm and approximately 100.0 cm in height is exposed.

The movable pulley 39 whose diameter is approximately 10.0 millimeters (mm) or larger, for example, is hung from a lower end of the U-shaped cable 32. The movable pulley 39 prevents the cable 32 from bending with the curvature smaller than the radius (for example, approximately 5.0 mm or over) of the movable pulley 39.

The movable pulley 39 is supported in a vertically movable manner, by a holder 39a, on a rail 40 fixed at right angles to the back surface of the cable receiving case 34 in the cable receiving space 34a. Also, the movable pulley 39 is urged downwards by a weight 41. The weight 41 provides a force to smoothly pull the cable 32 into the cable receiving space 34a.

Further, a cable lock 42 is installed in the movable pulley 39. The cable lock 42 consists of a rotatable lever-like member. With the use of such a cable lock 42, the disconnection of the cable 32 from the movable pulley 39 is prevented, improving the stability and reliability.

A movable-pulley lock 43 is installed at a position of approximately 70.0 cm in height of the rail 40 from the floor F. The movable-pulley lock 43 is a seesaw-like member that moves in a seesaw-like manner to change its position. With the use of the movable-lock 43, as will be described later, it is possible to hold the movable pulley 39 from which the cable 32 has been removed at a position equal to or higher than approximately 70.0 cm from the floor F, thereby making it easier to attach and detach the cable 32 to and from the movable pulley 39.

FIGS. 14 to 16 show steps to release the cable 32 from the movable pulley 39. In order to release the cable 32 from the movable pulley 39, first, the cover 36 is slid downwards. In a state where the inner portion of the cable receiving case 34 between 65 cm and 100 cm in height is exposed, the cable 32 is pulled out. Further, as shown in FIG. 14, the movable pulley 39 is raised to a position above the movable-pulley lock 43.

Then, as shown in FIG. 15, the movable-pulley lock 43 is made to project above an upper face 40a of the rail 40 by moving it in a seesaw-like manner, the movable pulley 39 is lowered, and the movable pulley 39 is mounted on the movable-pulley lock 43. Next, as shown in FIG. 16, the cable lock 42 is rotated and raised to release the cable 32 from the movable pulley 39.

Steps to hang the movable pulley 39 from the cable 32 are carried out in a reversed manner.

In the ultrasonic diagnostic apparatus 30 of the present embodiment described above also, the table 6, the image display device 4, and the processing device 5 are provided separately from the operating device 2. Further, the table 6, the image display device 4, and the processing device 5 are provided integrally on the side where the operating device 2 is not placed with respect to the table 6. Therefore, as in Embodiments 1 and 2, the space in the room in which the ultrasonic diagnostic apparatus 30 is installed can be used effectively.

In the present embodiment, the processing device 5 is provided above the cable receiving case 34 whose portion equal to or lower than approximately 100.0 cm from the floor F is approximately 10.0 cm thick or less. Therefore, the spacing between the table 6 which is lower than approximately 10.0 cm and the wall W can be set to approximately 11.0 cm, for example, allowing the table 6 to be positioned as close as possible to the wall W.

Also, according to the ultrasonic diagnostic apparatus 30 of the present embodiment also, the same advantages as other advantages in Embodiments 1 and 2 can be obtained.

Further, in the cable receiving case 34, the opening 34b is provided at a position equal to or higher than approximately 120.0 cm from the floor F. Thus, the cable 32 goes out and comes in at a position sufficiently higher than that of the patient on the table 6, which prevents the cable 32 from giving the patient discomfort.

Further, it is possible to receive the cable 32 inside the cable receiving case 34. Therefore, the cable 32 can be prevented from being in the way.

So far, the invention has been described with reference to the above embodiments. Needless to say, the invention can be embodied in variously modified manners without departing from the spirit thereof. For example, in Embodiment 2, though not particularly shown, the column support 21 and the processing device 5 may be integrated. Alternatively, in Embodiment 3, though not particularly shown, the column support 21, the cable receiving unit 33, and the processing device 5 may be integrated.

As for the processing device 5, the column support 21, or the cable receiving unit 33, when having a portion lower than the table 6 in terms of height from the floor F, it will do if the thickness of such a portion is approximately 10.0 cm or less. In the case where the mounting platform 13 of the table 6 goes up and down, when the mounting platform 13 is at the uppermost position, it will do if a portion of the processing device 5, the column support 21, or the cable receiving unit 33 lower than the table 6 has a thickness of approximately 10.0 cm or less.

Many widely different embodiments of the invention may be configured without departing from the spirit and the scope of the present invention. It should be understood that the present invention is not limited to the specific embodiments described in the specification, except as defined in the appended claims.

1. An ultrasonic diagnostic apparatus, comprising:
an operating device for an operator to input an instruction;
an image display device configured to display an ultrasonic image;
a processing device configured to produce the ultrasonic image based on an echo signal obtained by sending and receiving a ultrasonic wave and displaying it; and
a table on which a patient lies, wherein said operating device is provided separately from said image display device, said processing device, and said table, and wherein said table, said image display device, and said processing device are integrally provided on a side where said operating device is not placed with respect to said table.

2. An ultrasonic diagnostic apparatus according to claim 1, wherein, on said table, said processing device is provide on the side where said operating device is not placed and said image display device is provided at said processing device.

3. An ultrasonic diagnostic apparatus according to claim 1, wherein, on said table, said image display device and said processing device are provided on the side where said operating device is not placed.

4. An ultrasonic diagnostic apparatus according to claim 1, wherein said processing device comprises a portion whose height from a floor is lower than said table and a thickness of said portion is approximately 15.0 centimeters or less.

5. An ultrasonic diagnostic apparatus according to claim 3, wherein said image display device is provided at said table via a mounting part, and wherein each of said mounting part and said processing device comprises a respective portion whose height from a floor is lower than said table and a thickness of said portion is approximately 15.0 centimeters or less.

6. An ultrasonic diagnostic apparatus according to claim 1, wherein a probe configured to send and receive ultrasonic waves and said processing device each comprise a respective radio communication unit for mutual radio communication.

7. An ultrasonic diagnostic apparatus according to claim 1, wherein a probe body of the probe configured to send and receive ultrasonic waves and said processing device are connected via a cable and a cable receiving section for receiving said cable is provided on the side where said operating device is not provided with respect to said table.

8. An ultrasonic diagnostic apparatus according to claim 7, wherein said cable receiving section comprises a cable receiving case having a cable receiving space therein and being integral with said table, and wherein said cable receiving case comprises a portion whose height from a floor is lower than said table and a thickness of said portion is approximately 15.0 centimeters or less.

9. An ultrasonic diagnostic apparatus according to claim 8, wherein said processing device is integrally provided in an upper portion of said cable receiving case.

10. An ultrasonic diagnostic apparatus according to claim 1, wherein a shape of said table is changeable.

11. An ultrasonic diagnostic apparatus according to claim 2, wherein a shape of said table is changeable.

12. An ultrasonic diagnostic apparatus according to claim 3, wherein a shape of said table is changeable.

13. An ultrasonic diagnostic apparatus according to claim 4, wherein a shape of said table is changeable.

14. An ultrasonic diagnostic apparatus according to claim 6, wherein a shape of said table is changeable.

15. An ultrasonic diagnostic apparatus according to claim 7, wherein a shape of said table is changeable.

16. An ultrasonic diagnostic apparatus according to claim 8, wherein a shape of said table is changeable.

17. An ultrasonic diagnostic apparatus according to claim 9, wherein a shape of said table is changeable.

18. An ultrasonic diagnostic apparatus according to claim 10, wherein said table is configured to change between a shape of a bed and a shape of a chair.

19. An ultrasonic diagnostic apparatus according to claim 1, wherein said table comprises a height-adjusting part configured to adjust a height of a mounting platform, from a floor, of said table on which the patient lies.

20. An ultrasonic diagnostic apparatus according to claim 1, wherein said table comprises a grip part which the patient holds onto.
